(19) United States
(12) Patent Application Publication
(10) Pub. No.: US 2009/0221538 A1
(43) Pub. Date: Sep. 3, 2009

(54) METHODS OF TREATING MULTIPLE SCLEROSIS BY ADMINISTERING PULSE DOSE CALCITRIOL

(76) Inventors: Colleen E. Hayes, Madison, WI (US); Richard Derks, Cottage Grove, WI (US); Faye E. Nashold, Sun Prairie, WI (US)

Correspondence Address: Wisconsin Alumni Research Foundation (WARF), C/O Foley & Lardner LLP, Verex Plaza, 150 East Gilman Street, Madison, WI, 53703-1481, US

(21) Appl. No.: 12/364,364

(22) Filed: Feb. 2, 2009

Related U.S. Application Data
(60) Provisional application No. 61/025,338, filed on Feb. 1, 2008.

(51) Int. Cl. A61K 31/593; A61P 37/00

(52) U.S. Cl. 514/168; 514/167

(57) ABSTRACT

Prophylactic or therapeutic treatment to inhibit the development or progress of multiple sclerosis symptoms is provided by providing intermittently administered elevated doses of calcitriol, sufficiently infrequently to avoid hypercalcemia. Such methods may include maintaining at least about a normal blood level of vitamin D₃ as evidenced by a 25-(OH)D₃ level of at least about 50 nmol/L.

### CROSS-REFERENCE TO RELATED APPLICATIONS

This application claims priority to provisional application No. 61/025,338 filed Feb. 1, 2008, which application is incorporated herein in its entirety.

### TECHNICAL FIELD

The field of this invention is the prophylactic and therapeutic treatment of multiple sclerosis. More particularly, the field relates to the use of intermittent or pulsed dosing of calcitriol for the treatment of multiple sclerosis.

### BACKGROUND

Multiple sclerosis (MS) is a neurodegenerative disease characterized by focal destruction of myelin, axonal injury and loss, oligodendrocyte loss, and reactive astrocyte formation. T cell accumulation at the margins of chronic active MS lesions, and mononuclear cells in the perivascular spaces, suggest the generally-accepted hypothesis that neurodegeneration is secondary to autoimmune-mediated central nervous system (CNS) damage (1). Peripherally-activated, autoreactive T cells are thought to migrate into the CNS and initiate neuroinflammation by releasing pro-inflammatory cytokines and chemokines that recruit and activate additional inflammatory cell types. The MS lesions form as activated microglia flood the surrounding tissue with harmful inflammatory mediators, oxidizing free radicals, pro-apoptotic factors, and matrix-degrading proteases. MS often shows a relapsing-remitting course that is poorly understood. Genetic epidemiological studies have indicated a strong genetic basis for MS (2). However, high monozygotic twin discordance rates (approximately 75%) suggest that environmental risk factors determine whether MS develops in genetically-susceptible individuals.

A safe, effective, inexpensive MS therapeutic is urgently needed, because there is no cure or universally effective treatment for this chronic disease. MS affects ˜0.4 million Americans. Lifetime direct and indirect costs are ˜$2.2 million/patient. The FDA-approved, self-injectable MS drugs, interferon-beta-1 (Betaseron, Avonex, and Rebif) and glatiramer acetate (Copaxone), are expensive (˜$2000/mo), and relatively ineffective. They reduced the relapse rate ˜35% in some relapsing-remitting MS (RRMS) (3-5), but did not slow disability progression in RRMS, primary progressive MS (PPMS), or secondary progressive MS (SPMS) patients. Natalizumab (Tysabri), an integrin-specific monoclonal antibody (mAb), slowed disability progression ˜42% over two years (6), but increased the risk of cardiac problems and fatal progressive multifocal leukoencephalopathy. In view of these drawbacks, recent research has explored vitamin D₃ and its active metabolite, calcitriol as a treatment for MS.

Research into calcitriol was undertaken upon recognition of the very strong inverse correlation (r>−0.9) between MS disease prevalence and ultraviolet B light (UVB) exposure (7-10). Calcitriol (also known as 1,25-dihydroxy vitamin D₃ or 1,25-(OH)₂D₃) is unique among hormones in that its synthesis requires sunlight exposure (12). UVB photons catalyze the formation of vitamin D₃ from 7-dehydrocholesterol in the skin. (See below.)

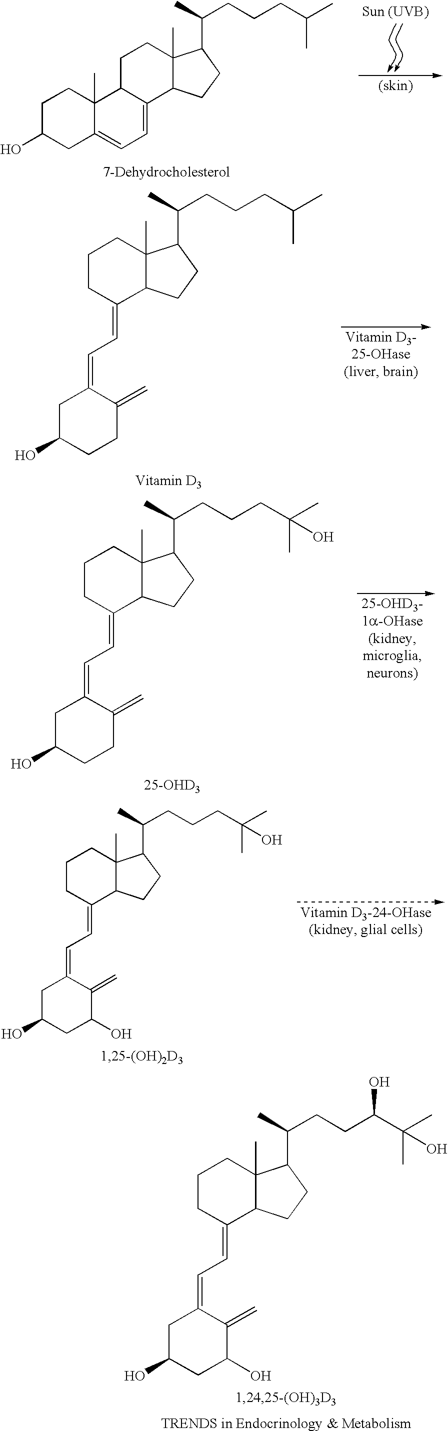

The vitamin D₃ is transported to the liver, where the enzyme vitamin D₃ 25-hydroxylase produces 25-(OH)D₃. This inactive vitamin D₃ metabolite is widely used as an indicator of vitamin D₃ supplies. The enzyme 25-hydroxyvitamin-D₃-1α-hydroxylase (1α-OHase) converts a small fraction of 25-(OH)D₃ into 1,25-(OH)₂D₃. The 1,25-(OH)₂D₃ is a highly active steroid hormone whose serum concentration is very tightly regulated by 1,25-(OH)₂D₃-mediated feedback inhibition of 1α-OHase activity, and 1,25-(OH)₂D₃-mediated induction of 1,25-dihydroxyvitamin D₃-24-hydroxylase (24-OHase) activity. The 24-OHase converts 1,25-(OH)₂D₃ into biologically inactive 1,24,25-trihydroxyvitamin D₃, which is further metabolized and excreted. Experimental autoimmune encephalomyelitis (EAE) is a recognized animal model for MS. Immunizing mice with neuroantigens like myelin basic protein (MBP) or myelin oligodendrocyte protein (MOG) induces EAE, a paralytic autoimmune disease with strong similarities to MS (13). The clinical EAE signs are ascending paralysis beginning with a loss of tail tone (stage 1) and progressing to hind- and fore-limb paralysis (stage 4). EAE can occur in relapsing-remitting or progressive forms. The primed, auto-reactive CD4⁺ T cells encounter CNS antigens presented on hematopoietically-derived, radio-sensitive antigen-presenting cells in the perivascular space, become re-activated, invade the brain parenchyma, and initiate an autoimmune reaction. The auto-reactive CD4⁺ T cells produce chemokines that attract monocytes, and pro-inflammatory cytokines (e.g. interleukin-17, interferon-γ, tumor necrosis factor-α) that activate the parenchymal microglia and cause reactive astrocyte formation. The activated macrophages and microglia produce cytokines that activate T cells (e.g. interleukin-12, interleukin-23) and neurotoxic chemicals that cause demyclination and axonal damage.

Research has shown that results obtained using EAE can be predictive for human treatments. For example, calcitriol (a metabolite of vitamin D₃) strongly inhibited EAE disease induction (14-18), prevented progression (15), and reversed established disease (17). These and other results led to a small clinical trial testing the hypothesis that calcitriol would inhibit MS (11). This important trial showed that daily calcitriol treatments decreased the MS relapse rate from an average of 1.0/yr to 0.2/yr, and completely inhibited MS progression. However, 2 of the 15 patients withdrew from the trial with symptomatic hypercalcemia. This adverse outcome highlights the well-known and potentially fatal risk that accompanies long-term daily calcitriol administration. Regrettably, this hypercalcemia risk prevented calcitriol from becoming an accepted MS therapeutic.

The following references identify the numbers indicated above.
1. Platten, M., and L. Steinman. 2005. Multiple sclerosis: trapped in deadly glue. Nature Medicine 11:252-253.
2. Oksenberg, J. R., S. E. Baranzini, L. F. Barcellos, and S. L. Hauser. 2001. Multiple sclerosis: genomic rewards. Journal of neuroimmunology 113:171-184.
3. 1993. Interferon beta-1b is effective in relapsing-remitting multiple sclerosis. I. Clinical results of a multicenter, randomized, double-blind, placebo-controlled trial. The IFNB Multiple Sclerosis Study Group. Neurology 43:655-661.
4. 1998. Randomised double-blind placebo-controlled study of interferon beta-1a in relapsing/remitting multiple sclerosis. PRISMS (Prevention of Relapses and Disability by Interferon beta-1a Subcutaneously in Multiple Sclerosis) Study Group. Lancet 352:1498-1504.
5. Johnson, K. P., B. R. Brooks, J. A. Cohen, C. C. Ford, J. Goldstein, R. P. Lisak, L. W. Myers, H. S. Panitch, J. W. Rose, and R. B. Schiffer. 1995. Copolymer 1 reduces relapse rate and improves disability in relapsing-remitting multiple sclerosis: results of a phase III multicenter, double-blind placebo-controlled trial. The Copolymer 1 Multiple Sclerosis Study Group. Neurology 45:1268-1276.
6. Polman, C. H., P. W. O'Connor, E. Havrdova, M. Hutchinson, L. Kappos, D. H. Miller, J. T. Phillips, F. D. Lublin, G. Giovannoni, A. Wajgt, M. Toal, F. Lynn, M. A. Panzara, and A. W. Sandrock. 2006. A randomized, placebo-controlled trial of natalizumab for relapsing multiple sclerosis. The New England journal of medicine 354:899-910.
7. Acheson, E. D., C. A. Bachrach, and F. M. Wright. 1960. Some comments on the relationship of the distribution of multiple sclerosis to latitude, solar radiation and other variables. Acta Psychiatry (Scandanavia) 35 (Supplement 147):132-147.
8. Hammond, S. R., D. R. English, and J. G. McLeod. 2000. The age-range of risk of developing multiple sclerosis: evidence from a migrant population in Australia. Brain 123:968-974.
9. van der Mei, I. A., A. L. Ponsonby, T. Dwyer, L. Blizzard, R. Simmons, B. V. Taylor, H. Butzkueven, and T. Kilpatrick. 2003. Past exposure to sun, skin phenotype, and risk of multiple sclerosis: case-control study. Bmj 327:316.
10. Goldacre, M. J., V. Seagroatt, D. Yeates, and E. D. Acheson. 2004. Skin cancer in people with multiple sclerosis: a record linkage study. J Epidemiol Community Health 58:142-144.
11. Wingerchuk, D. M., J. Lesaux, G. P. Rice, M. Kremenchutzky, and G. C. Ebers. 2005. A pilot study of oral calcitriol (1,25-dihydroxyvitamin D3) for relapsing-remitting multiple sclerosis. J Neurol Neurosurg Psychiatry 76:1294-1296.
12. Prosser, D. E., and G. Jones. 2004. Enzymes involved in the activation and inactivation of vitamin D. Trends Biochem Sci 29:664-673.
13. Steinman, L. 2003. Optic neuritis, a new variant of experimental encephalomyelitis, a durable model for all seasons, now in its seventieth year. J Exp Med 197:1065-1071.
14. Lemire, J. M., and D. C. Archer. 1991. 1,25-dihydroxyvitamin D3 prevents the in vivo induction of murine experimental autoimmune encephalomyelitis. The Journal of clinical investigation 87:1103-1107.
15. Cantorna, M. T., C. E. Hayes, and H. F. DeLuca. 1996. 1,25-Dihydroxyvitamin D3 reversibly blocks the progression of relapsing encephalomyelitis, a model of multiple sclerosis. Proceedings of the National Academy of Sciences of the United States of America 93:7861-7864.
16. Nashold, F. E., K. A. Hoag, J. Goverman, and C. E. Hayes. 2001. Rag-1-dependent cells are necessary for 1,25-dihydroxyvitamin D(3) prevention of experimental autoimmune encephalomyelitis. Journal of neuroimmunology 119:16-29.
17. Nashold, F. E., D. J. Miller, and C. E. Hayes. 2000. 1,25-dihydroxyvitamin D3 treatment decreases macrophage accumulation in the CNS of mice with experimental autoimmune encephalomyelitis. Journal of neuroimmunology 103:171-179.
18. Mattner, F., S. Smiroldo, F. Galbiati, M. Muller, P. Di Lucia, P. L. Poliani, G. Martino, P. Panina-Bordignon, and L. Adorini. 2000. Inhibition of Th1 development and treatment of chronic-relapsing experimental allergic encephalomyelitis by a non-hypercalcemic analogue of 1,25-dihydroxyvitamin D(3). Eur J Immunol 30:498-508.

### SUMMARY OF THE INVENTION

A prophylactically and therapeutically safe and effective protocol for the treatment of multiple sclerosis employs intermittent or pulsed doses of a calcitriol-enhancing drug to provide transiently elevated blood levels of calcitriol, while avoiding prolonged elevated levels of calcitriol that induce hypercalcemia and/or hypercalciuria. The methods may further include maintaining a serum level of 25-(OH)D₃ of at least 50 nmol/L. The protocol is repeated therapeutically as needed by MS symptom appearance or more frequently as indicated by patient experience. The protocol is found to reduce multiple sclerosis symptoms, reduce the time to remission, extend the time to relapse, and reduce cumulative disability and other symptoms.

Thus, in accordance with one aspect, the invention provides methods for inhibiting the development or progress of multiple sclerosis in a patient having MS and/or susceptible to the disabilities of MS. The methods include administering a dose of calcitriol-enhancing drug intermittently to the patient in an amount sufficient to inhibit the development or progress of multiple sclerosis and less than an amount to induce hypercalcemia. In some embodiments of the methods, the dose of calcitriol enhancing drug is at least about 0.1 μg/kg of calcitriol, or is in the range of about 0.1 to about 2 μg/kg. Alternatively, the dose of calcitriol-enhancing drug provides at least about 0.25 nmol/L calcitriol in the patient's blood, e.g., the dose provides a Cₘₐₓ of at least about 0.25 nmol/L. In some embodiments, the calcitriol dose provides a range from about 0.25 nmol/L to about 12 nmol/L calcitriol in the patient's blood. The methods can further include maintaining the patient's blood level of 25-(OH)D₃ at least at about 50 nmol/L by exposure to sunlight or UVB light, by diet, or by administration of supplements to enhance the amount of vitamin D₃ in the blood. In some embodiments the patient's blood level of 25-(OH)D₃ is maintained in the range of about 85 nmol/L to about 120 nmol/L.

In another aspect, there are provided methods for inhibiting the occurrence of the symptoms of multiple sclerosis in a patient susceptible to brain lesions associated with multiple sclerosis. The methods include administering a calcitriol dose intermittently to such a patient in an amount sufficient to inhibit the progress of multiple sclerosis and less than an amount to induce hypercalcemia, while maintaining a blood level of at least about 50 nmol/L 25-(OH)D₃ in the patient. In the methods, the intermittent administration can be less frequently than weekly and more frequently than annually. In some embodiments of the methods, the drug is calcitriol and the dose provides at least about 0.25 nmol/L calcitriol in the patient's blood, or provides a calcitriol level ranging from about 0.25 nmol/L to about 12 nmol/L in the patient's blood.

In another aspect, the invention provides methods for inhibiting the progress of multiple sclerosis in a patient suffering from the disabilities of multiple sclerosis. The methods include administering intermittently a calcitriol dose in the range of about 0.1 to about 2 μg/kg to the patient in an amount sufficient to inhibit the progress of multiple sclerosis and less than an amount to induce hypercalcemia. In the methods, the calcitriol dose can provide at least about 0.25 nmol/L calcitriol in the patient's blood, or can provide a calcitriol level ranging from about 0.25 nmol/L to about 12 nmol/L in the patient's blood. In some embodiments of the methods, the intermittent administration is less frequently than weekly and more frequently than annually. In the methods, 25-(OH)D₃ may be administered to maintain a 25-(OH)D₃ blood level of at least about 50 nmol/L, or in the range of about 85 to 100 nmol/L. In some embodiments of the methods, calcitriol is administered at an oral dose of about 0.5 μg/kg, and supplementary vitamin D₃ is administered after said calcitriol to maintain a 25-(OH)D₃ blood level at least about 85 nmol/L. The calcitriol may be administered once every 5 to 10 days, or less frequently as disclosed herein.

In another aspect the invention provides various dosage forms of calcitriol-enhancing drugs for use in the treatment of MS at any of the dosages disclosed herein, including pulse dose form. Thus, for example, calcitriol may be used to prepare a composition (e.g., medicament) in a pulse dose form for the treatment of multiple sclerosis. In one example, the composition comprises from about 0.1 to 2 μg/kg of calcitriol (e.g., about 1.5 μg to about 300 μg of calcitriol) and may further include an amount of vitamin D₃ sufficient to maintain a 25-(OH)D₃ blood level at least about 50 nmol/L or at least about 85 nmol/L. The amount of calcitriol in such compositions typically provides at least about 0.25 nmol/L calcitriol in a patient's blood, or provides a range from about 0.25 nmol/L to about 12 nmol/L calcitriol in the patient's blood.

In another embodiment, the invention provides compositions comprising a calcitriol-enhancing drug and vitamin D₃ as a combined preparation for simultaneous, separate, or sequential use in the treatment of multiple sclerosis. In such compositions the calcitriol-enhancing drug is formulated in a dosage for intermittent administration. The calcitriol-enhancing drug and the 25-(OH)D₃ may be provided as a single composition, or separately as parts of a kit. In some compositions, the calcitriol-enhancing drug is calcitriol. Any and all amounts of calcitriol and vitamin D₃ disclosed herein may be used in such compositions.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1. A pulse dose of 1,25-(OH)₂D₃ reduced the clinical EAE symptoms but did not prevent the resumption of disease progression in mice with EAE. Chow-fed female B10.PL mice with a clinical EAE score of 2.3±0.3 (achieved on day 17.3±2.5 days post MBP immunization) were randomized to receive an injection of 0.1 mL of oil only as a placebo (n=8) (open circles), or 200 ng of 1,25-(OH)₂D₃ dissolved in 0.1 mL of oil (n=5) (filled circles). The arrow denotes the day of treatment. Clinical EAE disability was evaluated daily for 21 days post treatment. Shown is the mean±S.D. for each group. The Wilcoxon rank sum test was used to determine the significance of differences between the 1,25-(OH)₂D₃ and placebo-treated groups. A p≦0.05, denoted by the *, was considered significant.

FIG. 2. Evaluation of the 1,25-(OH)₂D₃ pulse dose needed to reduce the clinical EAE symptoms in mice with EAE. Chow-fed female (panel A) and male (panel B) B10.PL mice with a clinical EAE score of 2.3±0.3 were randomized into groups. Mice were injected with 0.1 mL of oil only as a placebo, or with 20, 40, 200, or 400 ng of 1,25-(OH)₂D₃ dissolved in 0.1 mL of oil. Clinical EAE disability was evaluated daily for 14 days post treatment. Shown is the mean±S.D. for each group. The group sizes are shown in Tables 2 and 3.

FIG. 3. A pulse dose of 1,25-(OH)₂D₃ was superior to methyl-prednisolone for reducing clinical EAE symptoms in mice with EAE, and for maintaining remission. Panel A: Comparison of equimolar 1,25-(OH)₂D₃ and methyl-prednisolone pulse doses for reducing clinical EAE symptoms. Chow-fed female and male B10.PL mice with a clinical EAE score of 2.3±0.3 were randomized to receive a placebo (open circles), or 0.5 nmol (females) or 1.0 nmol (males) of methyl-prednisolone (squares) or 1,25-(OH)₂D₃ (filled circles). Females received 200 ng of 1,25-(OH)₂D₃ or 180 ng of methyl-prednisolone. Males received 400 ng of 1,25-(OH)₂D₃ or 360 ng of methyl-prednisolone. Clinical EAE disability was evaluated daily for 14 days post treatment. Shown is the mean for each group. The group sizes are given in Table 4. Panel B: 1,25-(OH)₂D₃ treatment to sustain a methyl-prednisolone-induced remission. Chow-fed female and male B10.PL mice with a clinical EAE score of 2.3±0.3 were randomized to receive a placebo (open circles), or 200 mg/d of methyl-prednisolone (filled triangles) until the drug-treated mice achieved a remission (sustained 1.0 point decrease in disability). The methyl-prednisolone treatments were then stopped and a pulse dose of 1,25-(OH)₂D₃ was administered (indicated by the arrow; females 200 ng; males 400 ng) to the mice in remission. Clinical EAE disability was evaluated daily for 28 days beginning with the first day of methyl-prednisolone treatment.

FIG. 4. Weekly 1,25-(OH)₂D₃ pulse doses reduced the clinical EAE symptoms and prevented the resumption of disease progression in mice with EAE. Chow-fed female B10.PL mice with a clinical EAE score of 2.3±0.3 were randomized to receive a weekly injection of 0.1 mL of oil only as a placebo (open circles; n=8), or 200 ng of 1,25-(OH)₂D₃ dissolved in 0.1 mL of oil (filled circles; n=6). The arrow denotes the days of treatment. Clinical EAE disability was evaluated daily for 21 days after treatment began. Shown is the mean±S.D. for each group. The Wilcoxon rank sum test was used to determine the significance of differences between the 1,25-(OH)₂D₃ and placebo-treated groups. A p≦0.05, denoted by the *, was considered significant.

FIG. 5. A pulse dose of 1,25-(OH)₂D₃ reduced the clinical EAE symptoms and prevented the resumption of disease progression in mice with high serum 25-(OH)D₃ levels. Chow-fed male and female B10.PL mice with a clinical EAE score of 2.0±0.5 were randomized into two groups. One group (n=115) was injected with a placebo, gavaged with a placebo 1 day later, and fed a synthetic diet formulated to provide 0 μg/day of vitamin D₃. The other group (n=18) was injected with 200 ng of 1,25-(OH)₂D₃, gavaged with 5 μg of vitamin D₃ 1 day later, and fed a synthetic diet formulated to provide 1 μg/day of vitamin D₃. The arrow denotes the day of treatment. Each t denotes a death. The groups were significantly different from day 25 onward (Wilcoxon test; p<0.05).

FIG. 6. A pulse dose of 1,25-(OH)₂D₃ did deplete splenic T and B lymphocytes or monocytes in mice with EAE. B10.PL mice with a clinical EAE score of 2.3±0.3 were injected with 175 ng of 1,25-(OH)₂D₃ dissolved in 0.1 mL of oil. Two weeks post treatment, splenocytes were collected from these mice (Panel B) and from naïve control mice (Panel A). Three-color flow cytometric analysis of splenic B lymphocytes was performed. Shown is one representative sample for each group.

### DETAILED DESCRIPTION

Safe effective methods for the prophylactic or therapeutic treatment of multiple sclerosis (MS) employ pulsed or intermittent administration of calcitriol-enhancing drug to provide blood levels of calcitriol above the normal physiological range, but less than an amount that induces hypercalcemia. Such methods can include maintaining a blood level of at least 50 nmol/L 25-(OH)D₃.

Patients that may be treated in accordance with the present methods suffer from or are susceptible to the disabilities of MS in all of its various forms. Generally, the present methods inhibit the development or progress of MS in patients susceptible to the disabilities of MS, inhibit the occurrence of symptoms of MS in patients susceptible to brain lesions associated with MS, and/or inhibit the progress of MS in patients suffering from the disabilities of MS. More specifically, in the RRMS form, the methods may reduce the time to remission, reduce overall disability during remission, prevent relapse or extend the time to relapse, reduce overall disability during relapse, and/or prevent or delay the conversion to SPMS disease. In the PPMS form (and in the SPMS form), the methods may induce a remission, or prevent or slow the appearance of new disease symptoms (inhibit disease progression). Desirably, the patients will be free of other medically significant diseases, for example, sarcoidosis, hyperthyroidism or hypothyroidism, cancer, diabetes, cardiovascular disease, and the like.

In the present methods, the calcitriol enhancing drug can be, e.g., calcitriol, a prodrug producing calcitriol due to physiological processes, a drug that inhibits physiological processes that degrade or metabolize calcitriol, or combinations thereof. The dose of the calcitriol enhancing drug administered elevates the patient's blood level of calcitriol above the normal physiological range over a period of at least one day, but not more than two days. The dose can be oral, or parenteral, or intramuscular.

The manifestations of MS disease are highly variable between individuals, and variable for a specific individual at different times in the individual's disease course. Some MS patients have a relapsing-remitting disease course characterized by worsening and subsequent improvement in disabilities, with a high degree of individual variability as regards the disabilities and as regards the periods of relapse and remission. Some relapsing-remitting MS patients have frequent relapses, whereas others have infrequent relapses. Some MS patients have a progressive disease course characterized by unremitting worsening in disabilities, with a high degree of individual variability as regards the disabilities and as regards the rate of disability progression. Some MS patients have a relapsing-remitting disease course followed by a progressive disease course. Lesions in the brain and spinal cord are characteristic of MS and underlie the clinical manifestations of MS. Individuals show a high degree of variability in lesion type, location, number, and volume, and in how the lesions are manifested in disability symptoms. Furthermore, the blood level of calcitriol and of 25-(OH)D₃, and the response to administration of calcitriol and vitamin D₃ to increase the blood levels of calcitriol and 25-(OH)D₃, and the changes in the condition of the patient are also subject to individual variation. There are differences in the response to administration of calcitriol and vitamin D₃ between sexes, diseases other than MS, and other genetic and physiological differences that can play a role in the nature of the treatment. Therefore, there will be a high degree of personalized treatment for patients suffering from MS. However, in view of the guidance provided herein, the skilled practitioner may adjust the frequency, dosage values, ranges and blood levels of calcitriol-enhancing drug and/or other treatment parameters accordingly to obtain the desired result. In general, the values and ranges indicated will be appropriate to those who may have a propensity for MS, have some indication of the initiation of MS, but are not symptomatic, or are suffering with MS.

In the case of prophylactic treatment, that is, the individual may have a propensity for MS, for example, when the individual is a biological first degree relative of an MS patient but is not symptomatic for MS, pulse doses of calcitriol are desirably administered at a level that does not cause calcium debility (hypercalcemia and hypercalciuria), or reduce the existing level of serum 25-(OH)D₃ in the patient. The amount of calcitriol administered in a bolus will generally be sufficient in most individuals to raise the calcitriol blood level within 3-24 hours to at least about 60% above the upper end of the normal range of 0.05-0.16 nmol/L (21-67 pg/mL), or even at least about 200% above the individual's level, e.g., raising the calcitriol blood level to an amount in the range of about 0.25 to about 12 nmol/L (104-5000 pg/mL), or in the range of about 0.5 to about 6 nmol/L (208-2500 pg/mL), or in the range of about 1 to about 2.4 nmol/L (417-1000 pg/mL). The serum 25-(OH)D₃ level should be at least about 50 nmol/L and may range from about 50 to about 150 nmol/L, usually from about 85 to about 120 nmol/L, as a result of inducing the maintenance or elevation of serum 25-(OH)D₃, employing vitamin D₃ or UVB light or sunlight or other entity that supports the serum 25-(OH)D₃ level. Substantially elevated levels may be maintained, as long as adverse indications are not observed.

Intermittent or pulsed dosing of calcitriol-enhancing drug means administration less frequently than daily, and typically less frequently than once every 3, 4 or 5 days or more, so as to be less frequent than to induce hypercalcemia. For example, the frequency of administration of the calcitriol will usually be not more often than once every 5 to 10 days, such as not more often than once weekly. It may also be biweekly or monthly or longer, even annually, depending upon the response of the patient, and may be administered in accordance with a specific schedule or sporadically, while observing for any signs that there might be an early stage of MS. The dosage may reflect the frequency of administration. Maintenance of serum 25-(OH)D₃ may not require any special action, although it can be desirable to administer vitamin D₃ or other substance to maintain the blood level of 25-(OH)D₃. It may be advantageous to give a supplementary dose of vitamin D₃ shortly after administration of the calcitriol, usually within 12 to 48 hours, conveniently within 12 to 36 hours, where the dose will be in the range of about 400 to 1500 IU/kg. (In referring to dosages per kg, these may be translated into a patient of about 65 kg.) Thereafter, as may be indicated by the physiological status of the person, serum 25-(OH)D₃ blood levels may be maintained as indicated above.

Patients who have exhibited at least one of the initial symptoms of MS or have clinically definite MS are given calcitriol at a sufficient dose to improve the condition of the patient, as evidenced by a decrease in the severity of the symptoms or the disabilities (induction of a remission), and/or a decrease in the cumulative disabilities, and/or an increase in the rate at which the symptoms or disabilities lessen (shortening of the time to remission), and/or a decrease in the rate at which existing disabilities worsen or new symptoms or disabilities appear (prevention or decrease in relapses per year or slowing of disease progression) and/or a reduction in brain lesion number or volume, and/or a reduction in the rate of new brain lesion formation. Where the patient has clinically definite MS, one will usually have a record of relapses and remissions, one may have a record of magnetic resonance imaging (MRI) scans, or initiate such scans, and one may have a blood analysis of at least the calcitriol level, the 25-(OH)D₃ level, and the calcium level, and may also include analysis of the cerebral spinal fluid. Therefore, the treatment will depart from earlier experience with no other drugs or where other drug regimens have been employed. In many cases, other drugs may have been used, such as anti-inflammatory drugs, e.g. statins, particularly simvastatin (Vollmer, et al. The Lancet, 363, 1607-8), interferon-beta-1 (Jacobs, et al. 1997 Ann Neurol 42, 982), glatiramer acetate (Johnson, et al. 1995 Neurology 45:1268-1276), cyclophosphamide (Herndon, et al. 1993 Neurology 43, 910), mitoxantrone, methyl-prednisolone (in combination, Edan, et al. J Neurology Neurophys and Psychiatry 1997 62, 112-8), and the like.

The MS patient may be at the initiation of symptoms, in remission, in recent relapse, or in active progressive disability. Generally remissions will occur within 6 days of a calcitriol treatment and may occur sooner. Time to relapse is greatly extended by calcitriol treatment, where patients may not have a relapse as long as they are maintained on an active schedule of calcitriol treatments, prescribed based on past experience with the patient or other patients having the same or similar prognosis and physiology.

Dosage forms of calcitriol-enhancing drug suitable for intermittent administration are referred to herein as “pulse dose forms” and may include, e.g., a higher dose of calcitriol that a daily dosage form suitable for treating MS, but not so high as to cause hypercalcemia. Thus, e.g., the patient will be administered a dose of calcitriol to elevate the blood level, and generally the dose will be at least about 0.1 μg/kg, or in the range of about 0.1 to about 2 μg/kg, about 0.2 to about 1.5 or about 1.2 μg/kg, or even in the range of about 0.4 to about 0.8 μg/kg, the amount being monitored to determine a maintenance dosage and frequency and to avoid hypercalcemia based on the frequency of administration. The blood level of calcitriol achieved using the present methods is generally at least about 0.25 nmol/L and may range from about 0.25 nmol/L to about 12 nmol/L, from about 1 to about 2.4 nmol/L.

The vitamin D₃ blood concentration may be maintained as it existed prior to pulse dose administration of calcitriol or elevated beyond that amount during the course of the treatment. Vitamin D₃ administration may be initiated before administration of the calcitriol or concomitant with the administration of calcitriol and will frequently continue after the administration of calcitriol. An initial bolus of vitamin D₃, if given, will generally be in the range of about 200 to 2000 IU/kg, usually in the range of about 400 to 1500 IU/kg, while daily or other scheduled maintenance doses, if given, will generally be in the range of about 20 to 200 IU/kg, usually in the range of about 30 to 62 IU/kg. A supplementary dose of vitamin D₃ may be provided within 12 to 48 hours of the administration of the calcitriol and thereafter the vitamin D₃ may be maintained by UVB light or sunlight exposure or dietary components or dietary supplements, e.g. pills containing vitamin D₃ to provide the required amount. Generally, it may be desirable to maintain the patient's blood level of 25-(OH)D₃ at least at about 50 nmol/L, or in a range from about 50 nmol/L or about 85 nmol/L to about 120 nmol/L.

The calcitriol may be administered on a regular schedule or as needed. The particular regimen will be chosen in accordance with the patient, the response of the patient to the treatment, the ease with which the patient's symptoms may be monitored, etc. A regular schedule may involve weekly, biweekly, monthly, bimonthly or the like, as experience with the patient accrues. In the case of as needed, the worsening of old symptoms or the appearance of new symptoms such as sensory deficits (e.g. numbness etc.), or vision deficits (e.g. loss of acuity, double vision etc.), or movement deficits (e.g. difficulty with muscle strength and coordination, partial or complete loss of fingers, thumb, hand, arm, foot, leg etc.), or increases in existing brain lesion size and/or the appearance of new brain lesions would encourage prompt administration of calcitriol or other modality, as needed. In relapsing-remitting MS patients, after the worsening old symptoms or new symptoms have decreased to a pre-exacerbation level, calcitriol may be administered on a schedule for a limited period of time, followed by a return to as needed administration. In progressive MS patients who have un-remitting disease progression, calcitriol may be administered on a schedule for an indefinite period of time to slow the rate of disease progression.

Instead of calcitriol, other agents may be administered that provide for an elevated blood level of calcitriol. These agents include inducers of production of calcitriol, inhibitors of enzymes that metabolize calcitriol (e.g. ketoconazole), and prodrugs of calcitriol, e.g. 1α-hydroxyvitamin D₃, 1α-hydroxyvitamin D₂, or calcitriol esters of carboxylic acids. However, agents that indirectly enhance calcitriol blood levels may be less desirable as there is a hiatus between the time of administration and the availability of an enhanced amount of calcitriol. Alternatively, one may administer agents that enhance the level of the vitamin D receptor, so that lesser amounts of calcitriol may be effective. There may be situations where such agents are used in combination with calcitriol, which would allow for lower dosages of calcitriol.

Instead of calcitriol, biologically active vitamin D compounds which are equivalents of calcitriol (i.e., analogs) may be administered. Such compounds include, but are not limited to: 1,25-(OH)₂D₂, 19-nor-1α,25-dihydroxyvitamin D₂ (a.k.a. Paricalcitol or Zemplar), 1α,25-(OH)₂-20-epi-22-oxa-24,26,27-trihomovitamin D₃ (a.k.a. KH1060 or Lexacalcitol), 20-epi-1α,25-dihydroxyvitamin D₃ (a.k.a. MC1288), 19-nor-14,20-bisepi-23-yne-1,25-dihydroxyvitamin D₃ (a.k.a. TX527), and 1,25-(OH)₂-16-ene-vitamin D₃.

When administering calcitriol, the patient is monitored for symptoms of hypercalcemia and/or hypercalciuria. When calcitriol is administered in an intermittent pulse dosing schedule, there may be incidents of transient hypercalcemia that resolve without intervention in a day or two. However, the goal of the intermittent calcitriol pulse dosing schedule is to obtain a therapeutic benefit without causing persistent hypercalcemia and hypercalciuria, so patients may be monitored for serum and urinary calcium during the calcitriol treatment. The corrected serum calcium value is used to grade hypercalcemia [corrected calcium=serum calcium+(4−serum albumin)×0.8]. Grade 1 hypercalcemia is defined as greater than the upper limit of the reference range to 11.5 mg/dL. Grade 2 hypercalcemia is defined as >11.5 to 12.5 mg/dL. Grade 3 hypercalcemia is defined as >12.5 mg/dL to 13.5 mg/dL. Grade 4 hypercalcemia is defined as >13.5 mg/dL. Grade 1 hypercalcemia is mild, but calcitriol treatment should be suspended, calcium intake should be reduced, and serum calcium should be monitored until the serum calcium returns to within the normal reference range. Grade 2 hypercalcemia is moderate, but calcitriol treatment should be suspended, calcium intake should be reduced, and if the grade 2 or 3 hypercalcemia is confirmed on a repeat analysis, or if it is associated with serious hypercalcemic symptoms, or at the discretion of the attending physician, the condition should be promptly treated. Grade 3-4 hypercalcemia is severe, calcitriol treatment should be suspended, and at the discretion of the attending physician, the patient may require hospitalization for treatment and monitoring. Various procedures are known for treating hypercalcemia, e.g. reduction in calcium intake, increase in fluid intake, administration of diuretics, corticosteroids like prednisone, and the like.

The subject protocols can be used in conjunction with presently investigated therapies, using cladribine, laquinimod, fingolimid, dimethyl fumarate, atacicept, tamoxifen, raloxifene, and antibodies, such as natalizumab, daclizumab, alemtuzumab and rituximab. These other drugs may be employed prior to and/or after using the subject treatment or alternating with the subject treatment or concomitant with the subject treatment. Because of individual responses, each treatment would have to be investigated and the patient monitored as to response, until a consensus protocol is reached based on an understanding of the response to the treatment and the conditions that warrant a particular protocol.

On the other hand, certain drugs should be avoided in conjunction with calcitriol treatment. These drugs include digoxin, thiazide diuretics, bisphosphonates, bile resin binding drugs like cholestyramine, magnesium containing antacids, calcium supplements, chloroquine, and corticosteroids like prednisone and methyl-prednisolone.

An illustrative protocol employs the analysis of the patient's serum calcitriol and 25-(OH)D₃ levels. If the patient has ≦50 nmol/L of 25-(OH)D₃, then the patient is given calcitriol pulse doses, for example about 0.5 μg/kg±50%, on a regular schedule, for example once each week or as needed to provide a therapeutic benefit without causing hypercalcemia. If the patient has 50-85 nmol/L of 25-(OH)D₃, then the patient is given calcitriol pulse doses, for example about 0.5 μg/kg±50%, on a regular schedule, for example once each two weeks or as needed to provide a therapeutic benefit without causing hypercalcemia. If the patient has >85 nmol/L of 25-(OH)D₃, then the patient is given calcitriol pulse doses, for example about 0.5 μg/kg±50%, on a regular schedule, for example once each month or as needed to provide a therapeutic benefit without causing hypercalcemia. A supplement of vitamin D₃, for example 400 to 1500 IU/kg, may be given shortly before or after the calcitriol pulse dose to increase the serum 25-(OH)D₃ level and decrease the frequency or dosage of calcitriol treatments needed for the therapeutic benefit. The supplement may be adjusted as needed to provide, e.g., serum 25-(OH)D₃ level of at least about 50 nmol/L or even about 85 nmol/L to about 120 nmol/L. The calcitriol pulse doses may be given orally, or by intravenous infusion, or by intramuscular injection.

Usually the calcitriol dose will be in the range of 0.2 to 1.2 μg/kg (13 to 78 μg per 65 kg adult), more usually in the range of about 0.5 to 1.0 μg/kg (32 to 65 μg per 65 kg adult). For convenience, the calcitriol can be compounded as a solution intended for intravenous infusion or intramuscular injection. Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may also be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Alternatively, the calcitriol can be compounded as a pill or capsule at the appropriate dosage, namely the individual dosage indicated above. For the most part, the dosage form will have at least about 32 μg, usually at least about 50 μg, and may have 65 μg or more. The pill or capsule will usually include other physiologically inert ingredients, fillers or excipients, such as talc, stabilizers, surfactants, cellulosic materials, e.g. plant starches, methyl cellulose, etc., sugars, such as lactose, sucrose, mannitol, sorbitol, etc., polyvinyl pyrrolidone, agar, alginic acid and salts, etc. These additional ingredients will be used in their conventional amounts.

The dosage form will usually be available in a container, a vial or the like, and may be included in a kit. The container will include the required labeling and provide for the frequency of dosing, if a specific regimen is involved, potential side effects, drugs to be avoided, symptoms that may occur that warrant a doctor's attention, and such other information that is provided with drugs. The calcitriol may be included in a kit including vitamin D₃ dosage forms, e.g. pill, where the vitamin D₃ container will be labeled with the appropriate label indicating the frequency of dosing, if a specific regimen is involved. The vitamin D₃ dosage will be at the dose range indicated above.

The subject treatments result in rapid remissions during active episodes, long periods to relapse, if at all, reductions in exacerbations, severity of disability and cumulative disability, reduced levels of exacerbation, and inhibition of increase in brain lesions associated with inflammation and demyelination.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### Experimental

### Materials and Methods

### Mice

Breeding pairs of B10.PL-H2^{u}H2-T18^{a}/(73NS)/SnJ (hereafter B10.PL) mice were purchased from the Jackson Laboratory (Bar Harbor, Me.). The experimental B10.PL mice were either bred in the pathogen-free mouse colony at the University of Wisconsin Department of Biochemistry, or purchased from the Jackson Laboratory. Mice were housed at 23° C. with 40-60% humidity, 12 hr light-dark cycles, and ad libidum access to water. Unless stated otherwise, the mice were fed Lab Diet #5008 (PMI Nutrition International, Inc., Brentwood, Mo.) containing 0.33 μg/d vitamin D₃ and 1% calcium. Experiments used male and female mice aged 6-8 wks (age- and sex-matched within experiments). The University of Wisconsin College of Agricultural and Life Sciences Institutional Animal Care and Use Committee approved all of the experimental protocols.

### EAE Induction and Evaluation of Clinical Signs

Bovine myelin basic protein (Sigma Chemical Co., St. Louis, Mo.) was dissolved at 2 mg/mL in 0.1 N acetic acid and emulsified with Complete Freund's adjuvant containing heat-killed Mycobacterium tuberculosis H37 Ra solution (Difco). Each mouse was injected subcutaneously with 0.1 mL emulsion in each hind flank (400 micrograms of MBP per mouse). On the day of immunization and again 2 days later, 200 nanograms of pertussis toxin was injected into the peritoneum. Thereafter, clinical EAE signs were scored daily as follows: 0, normal; 1, limp tail; 1.5, partial paralysis of one hind limb; 2, partial paralysis of both hind limbs; 2.5 partial paralysis of one hind limb and complete paralysis of one hind limb; 3, paralysis of both hind limbs; 4, hind and fore limb paralysis; 5, moribund.

### Vitamin D₃ or 1,25-(OH)₂D₃ Treatment

The vitamin D₃ and 1,25-(OH)₂D₃ were obtained from Sigma Chemical Co. The vitamin D₃ was dissolved in safflower oil and stored in the dark at 5° C. The 1,25-(OH)₂D₃ was dissolved in 100% ethanol (1 mg/mL) and stored in the dark under nitrogen gas at −20° C. The concentration of 1,25-(OH)₂D₃ was determined by spectral analysis. Animals with stage 2.3±0.3 were randomized into treatment groups. The details of the treatments are given in the figure legends and table footnotes. The synthetic diet used in some experiments was formulated to contain all essential nutrients except vitamin D₃ and was prepared exactly as we described (Spach and Hayes, 2005, J. Immunol. 175:4119-4126). For experiments using supplementary vitamin D₃, the placebo group was fed the synthetic diet ad libidum without added vitamin D₃, whereas the vitamin D₃-supplemented group was fed the synthetic diet ad libidum with vitamin D₃ added in an amount to provide 1 μg/d, calculated based on a daily measured consumption of 4.0 g dry weight of diet per mouse. Fresh synthetic diet was provided three times per week. At several times during the study, mice were weighed and blood samples were obtained from the tail vein. At the conclusion of the study, the mice were euthanized, a blood sample was obtained, perfusion was done, and the spinal cords and optic nerves were removed. The spinal cords were flash frozen with liquid nitrogen and stored at −70° C. prior to 1,25-(OH)₂D₃ extraction. Alternatively, the spinal cords were divided into 6 equal sections, aligned vertically, snap frozen in O.C.T. compound (Sakura Finetek USA, Torrance, Calif.) and stored at −70° C. for histopathology. The blood was clotted, centrifuged, and the decanted serum was frozen at −70° C. prior to analysis.

### Histopathology

For histopathological evaluation, mice were euthanized and perfused with saline. The spinal cords were removed, divided into 6 equal segments, frozen in O.C.T. compound (Sakura Finetek U.S.A., Inc., Torrance, Calif.) and sectioned transversely (10 μm). The cryosections were fixed in 4% paraformaldehyde, stained with Gill's No. 3 hematoxylin and eosin Y (Sigma Diagnostics, St. Louis, Mo.), and examined using a Zeiss Axioskop microscope equipped with a Plan-Neofluar 20×/0.5 objective. Bright field images were acquired with AxioVision 3.0 software controlling an Axiocam digital camera. For the histopathology analysis, each of six sections/mouse was divided into quadrants, and each quadrant was scored in a blinded fashion as 0 or 1, based on the absence or presence, respectively, of infiltrating inflammatory cells. The histopathology score was recorded as the percentage of spinal cord quadrants that showed a readily identifiable inflammatory cell infiltrate.

### Serum Calcium Analysis

Blood was collected, clotted, and centrifuged (2,000×g for 10 min) at 6° C. The serum was decanted and stored at −20° C. The samples, standards, and buffer blanks (2 μL each) were aliquoted into duplicate wells of a 96-well plate. The calcium detection reagent was prepared according to the manufacturer's directions (Sigma Diagnostics. St. Louis, Mo.), and 0.25 mL was added to each well. The absorbance at 570 nm less the blank was measured 10 to 30 min later. The Ca⁺⁺ mmol/L serum was determined from a standard curve.

### Serum 25-(OH)D and 1,25-(OH)₂D₃ Analysis

The serum was extracted and the 25-(OH)D₃ (DiaSorin, Stillwater, Minn.) and calcitriol (Nichols Institute Diagnostics, San Juan Capistrano, Calif.) concentrations were determined in duplicate with radioimmunoassay kits according to the manufacturer's protocols. The spinal cords were first extracted with a chloroform-methanol-4% KCl in water (1:2:0.8 v/v) mixture to recover the vitamin D metabolites. The spinal cord extracts were then assayed in duplicate for 1,25-(OH)₂D₃. A spike-recovery control was performed with each 1,25-(OH)₂D₃ extraction by adding 100 pg 1,25-(OH)₂D₃ to a crushed spinal cord from a vitamin D₃-depleted mouse (fed-D diet for >28 days). The extraction was then completed, the 1,25-(OH)₂D₃ was assayed in duplicate, the percentage recovery was calculated, and a recovery correction factor was applied to the experimental data. The hormone recovery averaged 70±10%.

### Splenocyte Staining and FACS Analysis

For flow cytometric analysis, splenocytes were dissociated into ice-cold Hank's balanced salt solution with HEPES buffer, and RBC were depleted. Duplicate samples (10⁶ cells/sample) were stained (30-45 min on ice) with optimal amounts of the mAb conjugates in staining buffer (PBS pH 7.3 with 5% heat-inactivated FBS and 0.1% NaN₃). Reference samples stained with single-color mAb served as controls to select fluorescence gates and flow cytometer compensation. The fluorescent mAb conjugates used for this analysis were FITC-labeled mAb to CD4 (clone L3T4) and APC-labeled mAb to CD11b (clone Macla) from Southern Biotechnology, and PE-labeled mAb to CD8 (clone alpha) and biotin-labeled mAb to CD45R (B220) from Invitrogen. PerCP-Cy5.5 was from BD/Pharmingan. Stained samples were analyzed on a FACScalibur™ using CELLQuest™ software (BD Biosciences, Franklin Lakes, N.J.).

### Statistical Analysis

Individual mice were analyzed and the mean and SD were calculated for each group of mice. Experiments were repeated at least once. The group sizes are given in the figure legends and tables. The significance of differences between the group means was determined using the Mann-Whitney test (n≦16), Student's t-test (n>16), or Chi-squared test (binomial data); p<0.05 was considered significant.

### Results

The purpose of the experiments was to evaluate the therapeutic potential of vitamin D₃ and 1,25-(OH)₂D₃ in EAE, a well-established animal model for MS. We first evaluated vitamin D₃ for an effect on clinical disability in mice with EAE disease. Mice with a clinical EAE disability score of 1.5±0.5 were randomized to receive 0 or 1 μg/day of vitamin D₃ (14-15 mice/group). This amount of vitamin D₃ is three-times the amount of vitamin D₃ provided by standard laboratory mouse chow. At the end of the 28 day study, the vitamin D₃ supplemented mice had 8217 nmol/L of serum 25-(OH)D₃ compared to 12±5 nmol/L in the un-supplemented mice (p<0.001). However, the two groups did not differ significantly in mortality (9-14% both groups), peak clinical score (mean 2.8 to 3.2 both groups), or cumulative disability (sum of daily disability scores; mean 71±18 both groups). The serum calcium levels in the vitamin D₃-supplemented mice (9.6±1.5 mg/dL) and un-supplemented mice (9.8±0.7 mg/dL) were not significantly different at the end of the study. These data show that the vitamin D₃ significantly increased the serum 25-(OH)D₃ level without causing hypercalcemia, but had no effect on clinical disability in mice with EAE.

To circumvent the problems of hypercalcemia and hypercalciuria associated with daily long-term 1,25-(OH)₂D₃ administration, we tested the efficacy of intermittent 1,25-(OH)₂D₃ pulse dosing as a treatment for acute EAE.

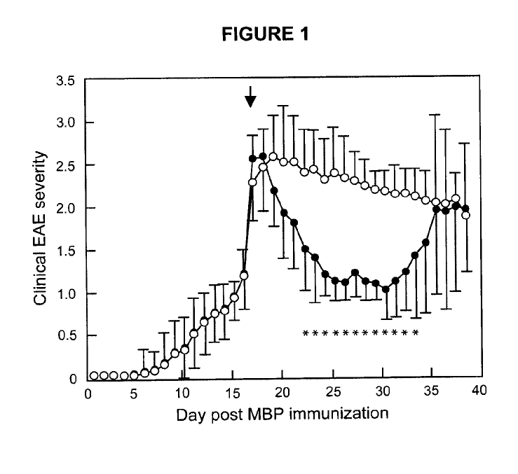

A pulse dose of 200 ng 1,25-(OH)₂D₃ rapidly induced a remission in female mice with acute EAE disease (FIG. 1 and Table 1). Animals in this study were fed standard laboratory chow and had 52±18 nmol/L of serum 25-(OH)D₃. Female mice were immunized with MBP to induce EAE. Mice with a clinical EAE score of 2.3±0.3 were randomized to receive an injection of oil only or 200 ng of 1,25-(OH)₂D₃ dissolved in oil, and evaluated daily thereafter for clinical EAE disability. The clinical disability scores of the 1,25-(OH)₂D₃-treated mice declined rapidly and significantly such that within 6 days, 100% of these mice achieved a remission, defined as EAE≦1.5 for at least two consecutive days. The remission lasted an average of 14 days before a relapse occurred. In sharp contrast, the clinical disability scores of the placebo-treated mice did not decline significantly. Only one of eight placebo-treated mice achieved a remission. The cumulative disability of the 1,25-(OH)₂D₃-treated mice was significantly lower than the placebo-treated mice during the 21 day observation period. Very similar results were obtained for male mice (data not shown). These data show that in mice with normal serum 25-(OH)D₃ levels, a pulse dose of 200 ng 1,25-(OH)₂D₃ rapidly ameliorated the clinical signs of acute EAE disease, but did not prevent a relapse.

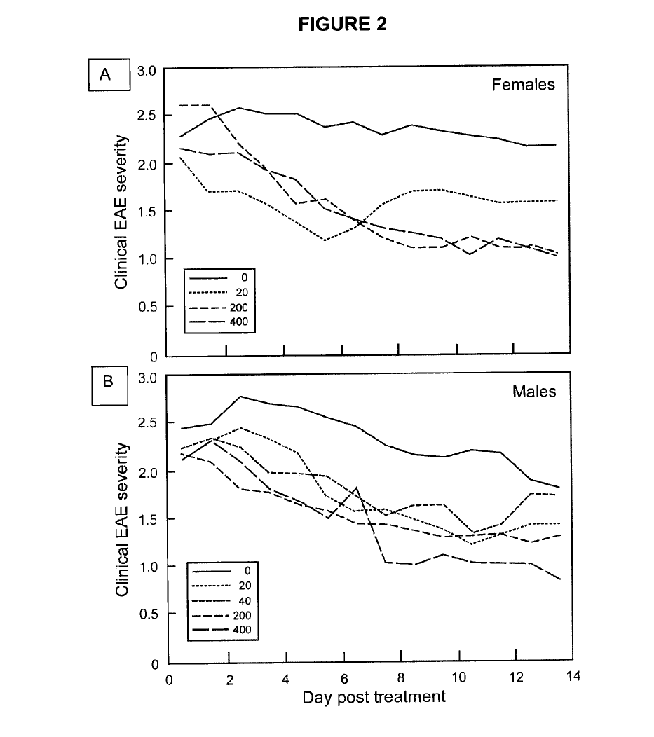

Further experiments tested a range of 1,25-(OH)₂D₃ pulse doses in female and male chow-fed mice with acute EAE disease. Female mice with MBP-induced EAE were randomized to receive an injection of oil only or 20, 200, or 400 ng of 1,25-(OH)₂D₃ dissolved in oil, and evaluated daily thereafter for clinical EAE disability. The 20 ng dose was partially effective for the treatment of EAE (FIG. 2 and Table 2). This dose significantly increased the percentage of mice achieving remission, shortened the time to remission, and decreased the cumulative disability over the 14 day observation period, but did not significantly increase the days in remission, or decrease the EAE disability 14 days post treatment. However, the 200 and 400 ng pulse doses significantly increased the percentage of mice achieving remission, shortened the time to remission, increased the days in remission, and decreased the EAE severity 14 days post treatment. Similarly in male mice with acute EAE disease, the 20, 40, and 200 ng pulse doses were suboptimal for at least one parameter relating to remission, whereas the 400 ng pulse dose significantly increased the percentage of mice achieving remission, shortened the time to remission, increased the days in remission, and decreased the EAE disability 14 days post treatment (FIG. 2 and Table 3). None of these 1,25-(OH)₂D₃ pulse doses prevented a resumption of EAE disease progression (FIG. 1 and data not shown). These results show that in mice with normal serum 25-(OH)D₃ levels, a 200 ng pulse dose in females and a 400 ng pulse dose in males optimally ameliorated EAE disease.

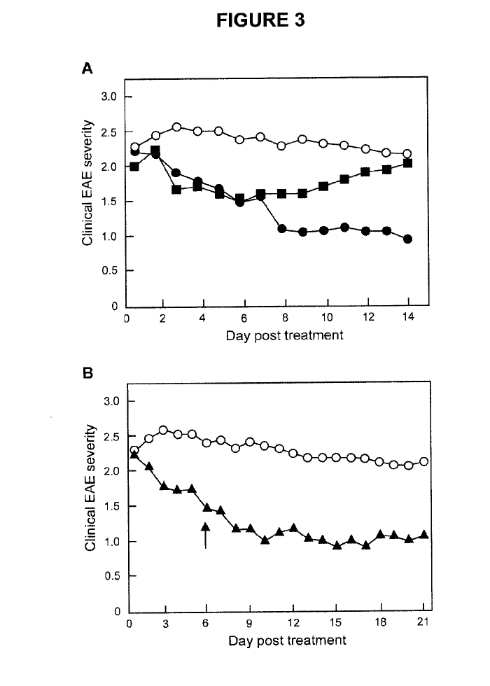

Methyl-prednisolone, a corticosteroid, is the treatment of choice for relapsing MS patients (Sloka and Stefanelli, 2005 Mult. Scler. 11:425). Corticosteroids are known to inhibit the synthesis of 1,25-(OH)₂D₃ (Sharma, 2000, Cur Opin Pulmon Med 6:442-447). Therefore, it was of interest to determine the relative potency of methyl-prednisolone and 1,25-(OH)₂D₃ for the treatment of EAE disability. Chow-fed mice with EAE were randomized to receive a placebo, or 0.5 nmol (females) or 1.0 nmol (males) of methyl-prednisolone or 1,25-(OH)₂D₃. The 1,25-(OH)₂D₃ treatment significantly increased the percentage of mice achieving a reduction in disability, shortened the time to remission, increased the days in remission, and decreased the cumulative disability compared to the placebo treatment (FIG. 3A). In sharp contrast, the 0.5 nmol (females) or 1.0 nmol (males) methyl-prednisolone treatment shortened the time to remission in the 40% of mice achieving remission, but the remissions were short-lived. Consequently, the slight trend towards lower cumulative disability in the methyl-prednisolone-treated animals did not reach significance (Table 4). These results show that the 1,25-(OH)₂D₃ is far superior to methyl-prednisolone for the treatment of acute EAE disability.

Others reported that daily oral administration of 200 μg/d (0.5 μmol/d) of methyl-prednisolone to mice with EAE induced a temporary remission, but 100% of the mice relapsed within 3-5 d when the drug was withdrawn (Chan et al. 2008 Autoimmunity 41:405-413). It was of interest to determine whether 1,25-(OH)₂D₃ treatment could prolong a methyl-prednisolone-induced remission. Chow-fed mice with EAE (score 2.3±0.4) were randomized to receive placebo or 200 μg/d of oral methyl-prednisolone. This high methyl-prednisolone treatment induced remission in 100% of the male and female mice after 6±2 days (FIG. 3B). The methyl-prednisolone was then withdrawn and one 1,25-(OH)₂D₃ treatment was administered (females 200 ng; males, 400 ng). In contrast to the 100% rapid relapses reported by Chan et al. (ibid) when methyl-prednisolone treatment was withdrawn, 4 of 6 mice treated with 1,25-(OH)₂D₃ did not relapse in a 28-day observation period. The remaining two mice relapsed after 14 or 23 days in remission, respectively. The cumulative disability in the 28-day observation period was 62±9 for the placebo group and 36±10 for the methyl-prednisolone followed by 1,25-(OH)₂D₃ treated group, a 42% reduction. These results show that the 1,25-(OH)₂D₃ treatment is effective for prolonging a methyl-prednisolone-induced EAE remission.

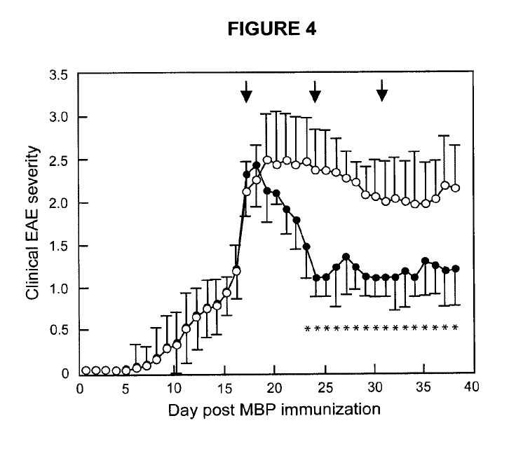

Because the remission induced by a single 1,25-(OH)₂D₃ pulse dose was followed by a relapse, it was of interest to determine the effect of multiple 1,25-(OH)₂D₃ pulse doses. Chow-fed mice with acute EAE were randomized to receive weekly injections of placebo or 200 ng of 1,25-(OH)₂D₃, and evaluated daily for clinical EAE disability. The weekly 1,25-(OH)₂D₃ treatments significantly increased the percentage of mice achieving remission, shortened the time to remission, increased the days in remission, and prevented disability progression (FIG. 4 and Table 2). The cumulative disability of the 1,25-(OH)₂D₃-treated mice was 43% lower than the placebo group. These data show that in mice with normal serum 25-(OH)D3 levels, weekly 1,25-(OH)₂D₃ treatments reduced EAE disability and prevented disease progression.

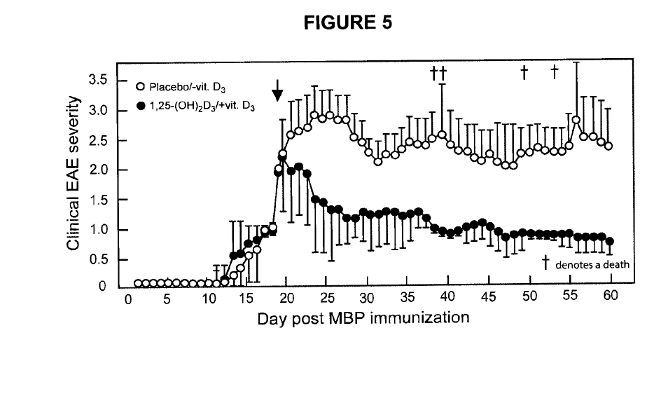

The single 1,25-(OH)₂D₃ pulse dose ameliorated acute EAE disease, but did not prevent a relapse in mice with normal serum 25-(OH)D₃ levels. In contrast, high levels of serum 25-(OH)D₃ did not inhibit established EAE disease. Therefore, it was of interest to evaluate the effect of a 1,25-(OH)₂D₃ pulse dose in mice with acute EAE, whose serum 25-(OH)D₃ levels were above the normal range for chow-fed mice. Mice with a clinical EAE score of 2.0±0.5 were randomized into two groups. One group was injected with a placebo, gavaged with a placebo, and fed a synthetic diet formulated to provide 0 μg/day of vitamin D₃. The serum 25-(OH)D₃ level in this group declined to 16±6 nmol/L at day 10 and to 10±4 nmol/L at day 28 post treatment. The other group was injected with 200 ng of 1,25-(OH)₂D₃, gavaged with 5 μg of vitamin D₃, and fed a synthetic diet formulated to provide 1 μg/day of vitamin D₃. The serum 25-(OH)D₃ level in this group rose to 82±27 nmol/L at day 10 and to 102±39 nmol/L at day 28 post treatment. Remarkably, in mice with 82±27 of serum 25-(OH)D₃, the 1,25-(OH)₂D₃ pulse dose induced a very long-term remission, reducing the cumulative disability 48% over the 42 day observation period with no subsequent resumption of disease progression (FIG. 5 and Table 5). The serum calcium levels were not significantly different between the two groups. At the end of the study, the 1,25-(OH)₂D₃-treated group had 18.8±6.6 pmol/mL of 1,25-(OH)₃D₃ in the spinal cord compared to 4.9±4.4 pmol/mL in the placebo group. These data show that in mice with 82±27 nmol/L of serum 25-(OH)D₃, the 1,25-(OH)₂D₃ pulse dose ameliorated EAE and prevented disease progression without a risk of hypercalcemia.

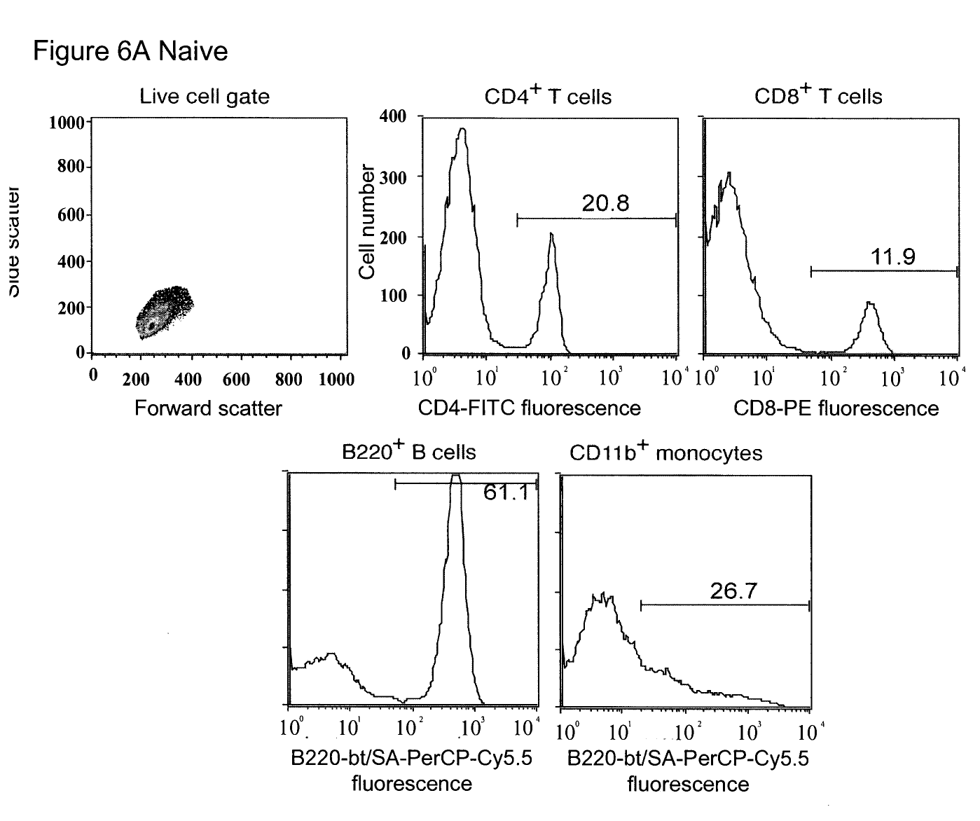

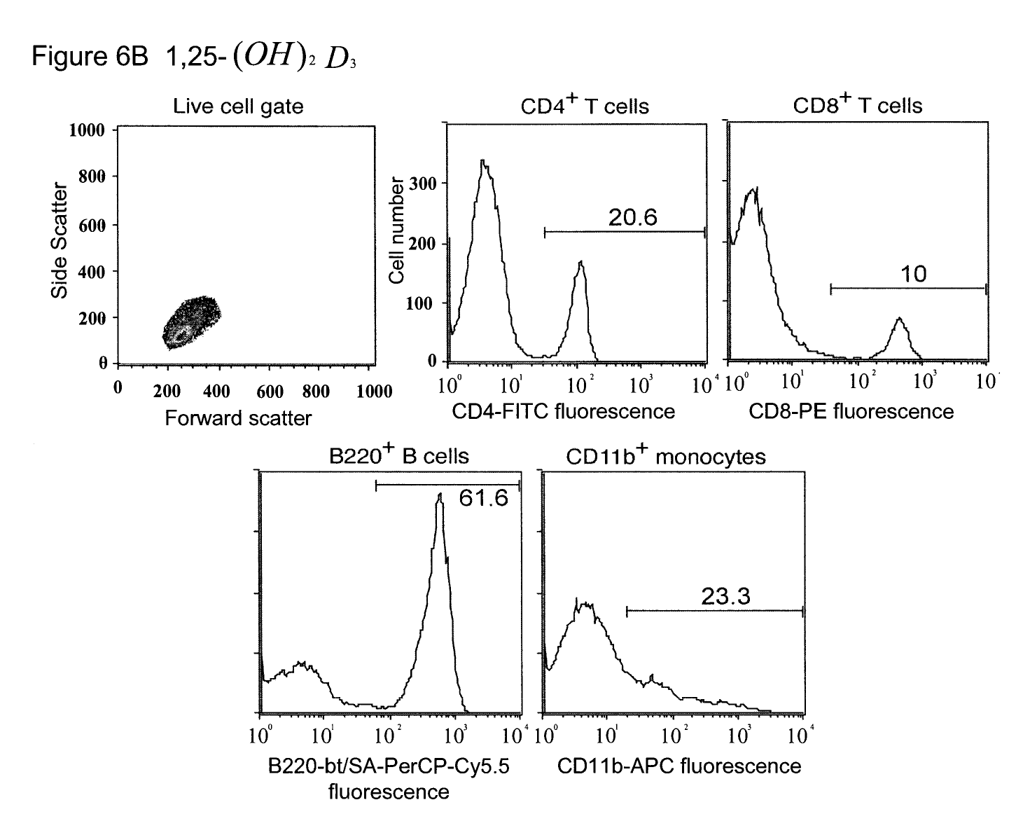

Several current MS therapies deplete lymphocytes and monocytes, resulting in an immunodeficient state and a significantly increased susceptibility to infectious disease (Wingerchuk, 2008, Semin Neurol 28:56). To determine whether a 1,25-(OH)₂D₃ pulse dose would result in an immunodeficient state, we collected splenocytes 14 days after a 175 ng 1,25-(OH)₂D₃ pulse dose and quantified lymphocytes and monocytes (FIG. 6). There were no significant differences between the 1,25-(OH)₂D₃-treated mice and normal control mice with respect to numbers of splenocytes recovered (43±9 million cells) or proportions of CD4⁺ T lymphocytes (18±1%), CD8⁺ T lymphocytes (11±1%), B220⁺ B lymphocytes (63±3%) or CD11b⁺ monocytes (18±2%). These results show that a 1,25-(OH)₂D₃ pulse dose did not result in an immunodeficient state through the depletion of lymphocytes and monocytes.

The following tables provide the results in table form as discussed in the above description of the results.

TABLE 1
The 1,25-(OH)₂D₃ treatment rapidly induced a remission in female B10.PL mice with
acute EAE.^{a}

| 1,25-(OH)₂D₃ | EAE severity | | Remission^{b} | | | Cumulative |
|---|---|---|---|---|---|---|
| (ng) | (initial) | (peak) | (%) | (minimum) | (days) | disability^{c} |
| 0 | 2.3 ± 0.5 | 2.9 ± 0.4 | 12 | 1.8 ± 0.3 | 1.8 ± 3.9 | 47.3 ± 6.0 |
| (n = 8) | | | | | | |
| 200 once | 2.6 ± 0.4 | 2.7 ± 0.4 | 100 | 1.0 ± 0.4 | 13.4 ± 3.9 | 32.9 ± 9.3 |
| (n = 5) | (n.s.) | (n.s.) | (p ≦ 0.01) | (p ≦ 0.001) | (p ≦ 0.01) | (p ≦ 0.01) |
| 200 weekly | 2.3 ± 0.5 | 2.4 ± 0.4 | 100 | 1.0 ± 0.0 | 15.7 ± 3.4 | 31.6 ± 5.1 |
| (n = 6) | (n.s.) | (n.s.) | (p ≦ 0.01) | (p ≦ 0.001) | (p ≦ 0.01) | (p ≦ 0.01) |

^{a}Female B10.PL mice ingesting a standard laboratory chow diet were immunized with MBP to induce EAE. Mice with a clinical EAE score of 2.3 ± 0.3 were randomized into groups. The groups were identical with respect to day of EAE disease onset (11 ± 1 days post MBP immunization), the day of treatment (17.3 ± 2.5 days post MBP immunization), and the mortality (0% in all groups). Mice were injected with 0.1 mL of oil only as a placebo, or with 200 ng of 1,25-(OH)₂D₃ dissolved in 0.1 mL of oil. The dose was administered once or once each week. Clinical EAE disability was evaluated daily for 21 days post treatment. Shown is the mean ± S.D. for each group. The Wilcoxon rank sum test was used to determine the significance of differences between the 1,25-(OH)₂D₃ and placebo-treated groups, except for remission incidence data which was subjected to a Chi square test. A p ≦ 0.05 was considered significant.
^{b}Remission was defined as a clinical EAE disability score ≦1.5 for two or more consecutive days. Relapse was defined as a clinical EAE disability score >2.0 for two or more consecutive days. The days to achieve remission was calculated only for mice that achieved remission. For the mice that did not achieve remission, a zero was entered as days in remission.
^{c}The cumulative disability was calculated as the sum of each animal's daily clinical scores for 21 days beginning with the day of treatment.

TABLE 2
The 1,25-(OH)₂D₃ treatment rapidly induced a remission in female B10.PL mice with acute EAE.^{a}

| 1,25- | | | | | | | |
|---|---|---|---|---|---|---|---|
| (OH)₂D₃ | Remission^{b} | | | EAE severity | | | Cumulative |
| (ng) | (%) | (days to) | (days in) | (initial) | (final) | (decrease) | disability^{c} |
| 0 | 12 | 7 | 0.5 ± 1.8 | 2.3 ± 0.5 | 2.2 ± 0.2 | 0.1 ± 0.5 | 32.9 ± 4.4 |
| (n = 8) | | | | | | | |
| 20 | 75 | 2.0 ± 0.0 | 8.0 ± 6.3 | 2.1 ± 0.1 | 1.6 ± 1.0 | 0.5 ± 1.0 | 22.1 ± 9.0 |
| (n = 4) | (n.s.) | (p ≦ 0.05) | (n.s.) | (n.s.) | (n.s.) | (n.s.) | (p ≦ 0.01) |
| 200 | 100 | 5.8 ± 2.5 | 9.2 ± 2.5 | 2.6 ± 0.4 | 1.0 ± 0.4 | 1.6 ± 0.4 | 21.8 ± 4.6 |
| (n = 5) | (p ≦ 0.01) | (p ≦ 0.01) | (p ≦ 0.001) | (n.s.) | (p ≦ 0.001) | (p ≦ 0.001) | (p ≦ 0.01) |
| 400 | 100 | 6.2 ± 3.3 | 9.6 ± 3.0 | 2.2 ± 0.2 | 1.0 ± 0.0 | 1.2 ± 0.2 | 20.6 ± 5.2 |
| (n = 5) | (p ≦ 0.01) | (p ≦ 0.01) | (p ≦ 0.001) | (n.s.) | (p ≦ 0.001) | (p ≦ 0.001) | (p ≦ 0.01) |

^{a}Chow-fed female B10.PL mice with EAE were randomized into groups as described in the Table 1 footnote. Mice were injected with 0.1 mL of oil only as a placebo, or with 20, 200, or 400 ng of 1,25-(OH)₂D₃ dissolved in 0.1 mL of oil. Clinical EAE disability was evaluated daily for 14 days post treatment. Shown is the mean ± S.D. for each group. The statistical analysis was performed as described in the Table 1 footnote. A p ≦ 0.05 was considered significant.
^{b}Remission and relapse were defined and evaluated as described in the Table 1 footnote.
^{c}The cumulative disability was calculated as the sum of each animal's daily clinical scores for 14 days beginning with the day of treatment.

TABLE 3
The 1,25-(OH)₂D₃ treatment rapidly induced a remission in male BIO.PL mice with
acute EAE.^{a}

| 1,25- | | | | | | | |
|---|---|---|---|---|---|---|---|
| (OH)₂D₃ | Remission^{b} | | | EAE severity | | | Cumulative |
| (ng) | (%) | (days to) | (days in) | (initial) | (final) | (decrease) | disability^{c} |
| 0 | 67 | 12.5 ± 3.8 | 2.8 ± 3.1 | 2.4 ± 0.5 | 1.8 ± 0.6 | 0.7 ± 0.7 | 32.2 ± 5.0 |
| (n = 6) | | | | | | | |
| 20 | 80 | 6.3 ± 2.2 | 6.6 ± 4.6 | 2.1 ± 0.2 | 1.4 ± 0.5 | 0.7 ± 0.4 | 24.2 ± 6.2 |
| (n = 5) | (n.s.) | (p ≦ 0.01) | (n.s.) | (n.s.) | (n.s.) | (n.s.) | (p ≦ 0.05) |
| 40 | 80 | 4.5 ± 1.7 | 8.0 ± 4.6 | 2.2 ± 0.4 | 1.7 ± 1.0 | 0.5 ± 1.2 | 25.0 ± 9.0 |
| (n = 5) | (n.s.) | (p ≦ 0.01) | (n.s.) | (n.s.) | (n.s.) | (n.s.) | (n.s.) |
| 200 | 100 | 6.3 ± 2.4 | 11.9 ± 2.0 | 2.3 ± 0.3 | 1.3 ± 0.4 | 1.0 ± 0.4 | 22.1 ± 3.5 |
| (n = 7) | (n.s.) | (p ≦ 0.02) | (p ≦ 0.001) | (n.s.) | (n.s.) | (n.s.) | (p ≦ 0.001) |
| 400 | 86 | 4.2 ± 1.7 | 11.0 ± 1.4 | 2.1 ± 0.4 | 0.8 ± 0.3 | 1.2 ± 0.2 | 16.9 ± 2.5 |
| (n = 7) | (n.s.) | (p ≦ 0.01) | (p ≦ 0.005) | (n.s.) | (p ≦ 0.01) | (p ≦ 0.02) | (p ≦ 0.01) |

^{a}Chow-fed male B10.PL mice with EAE were randomized into groups as described in the Table 1 footnote. Mice were injected with 0.1 mL of oil only as a placebo, or with 20, 40, 200, or 400 ng of 1,25-(OH)₂D₃ dissolved in 0.1 mL of oil. Clinical EAE disability was evaluated daily for 14 days post treatment. One animal died 6 days post treatment with 400 ng of 1,25-(OH)₂D₃ without achieving a remission; a score of 5 was recorded on the day of death and no score was entered thereafter. Shown is the mean ± S.D. for each group. The statistical analysis was performed as described in the Table 1 footnote. A p ≦ 0.05 was considered significant.
^{b}Remission and relapse were defined and evaluated as described in the Table 1 footnote.
^{c}The cumulative disability was calculated as the sum of each animal's daily clinical scores for 14 days beginning with the day of treatment.

TABLE 4
The 1,25-(OH)₂D₃ treatment was superior to methyl-prednisolone
treatment for inducing a remission in B10.PL mice with acute EAE.^{a}

| | EAE severity | | Remission^{c} | | | Cumulative |
|---|---|---|---|---|---|---|
| Treatment^{b} | (initial) | (decrease) | (%) | (days to) | (days in) | disability^{d} |
| Placebo | 2.3 ± 0.5 | 0.6 ± 0.5 | 36 | 11.4 ± 4.1 | 2.0 ± 3.4 | 32.6 ± 4.5 |
| (n = 14) | | | | | | |
| 1,25-(OH)₂D₃ | 2.3 ± 0.4 | 1.2 ± 0.6 | 92 | 4.9 ± 2.2 | 10.0 ± 2.2 | 19.7 ± 4.4 |
| (n = 12) | (n.s.) | (n.s.) | (p ≦ 0.02) | (p ≦ 0.001) | (p ≦ 0.001) | (p ≦ 0.0001) |
| Methyl- | 2.0 ± 0.0 | 0.8 ± 0.7 | 40 | 3.0 ± 0.0 | 3.0 ± 4.1 | 24.8 ± 6.9 |
| prednisolone | (n.s.) | (n.s.) | (n.s.) | (p ≦ 0.001) | (n.s.) | (n.s.) |
| (n = 5) | | | | | | |

^{a}Chow-fed female and male B10.PL mice with EAE were randomized into groups as described in the Table 1 footnote. Mice were injected with 0.1 mL of oil only as a placebo, or with 1,25-(OH)₂D₃ or methyl-prednisolone dissolved in 0.1 mL of oil. Clinical EAE disability was evaluated daily for 14 days post treatment. Shown is the mean ± S.D. for each group. The statistical analysis was performed as described in the Table 1 footnote. A p ≦ 0.05 was considered significant.
^{b}The mice received 0.5 (female) or 1.0 (male) nmol of steroid compound as treatment. Females received 200 ng of 1,25-(OH)₂D₃ or 180 ng of methyl-prednisolone. Males received 400 ng of 1,25-(OH)₂D₃ or 360 ng of methyl-prednisolone.
^{c}Remission and relapse were defined and evaluated as described in the Table 1 footnote.

TABLE 5
One 1,25-(OH)₂D₃ treatment plus supplementary vitamin D₃ induced a long-term
remission in male and female B10.PL mice with acute MBP-induced EAE.^{a}

| | Dietary | Serum | Serum | | | | |
|---|---|---|---|---|---|---|---|
| 1,25-(OH)₂D₃ | vit. D₃ | 25-(OH)D₃ | Ca | Mortality | Remission^{b} | | Cumulative |
| (ng) | (μg/d) | (nmol/L) | (mg/dL) | (%) | (%) | (days) | disability^{c} |
| 0 | 0 | 12 ± 5 | 9.8 ± 0.7 | 27 | 0 | 0 ± 0 | 96 ± 9 |
| 200 | 1 | 82 ± 27* | 9.5 ± 1.2 | 0 | 100* | 36 ± 6* | 50 ± 14* |

^{a}Chow-fed female and male B10.PL mice with EAE were randomized into groups as described in the Table 1 footnote. One group (n = 15) was injected with a placebo, gavaged with a placebo 24 hr post injection, and fed a synthetic diet formulated to provide 0 μg/day of vitamin D₃. The other group (n = 18) was injected with 200 ng of 1,25-(OH)₂D₃, gavaged with 5 μg of vitamin D₃ 24 hr post injection, and fed a synthetic diet formulated to provide 1 μg/day of vitamin D₃. Shown is the mean ± S.D. for one experiment of two. The * indicates p < 0.001 (Student's t test).
^{b}Remission and relapse were defined and evaluated as described in the Table 1 footnote.
^{c}The cumulative disability was calculated as the sum of each mouse's daily clinical scores for the first 42 days post treatment; shown is the mean ± S.D. cumulative disease index for each group of mice.

Benefits of the 1,25-(OH)₂D₃ (calcitriol) pulse doses include increased rate of remission, reduced time to remission, increased time to relapse, decreased rate of exacerbation, reduced daily disability, reduced cumulative disability, prevention of progressive increases in disability, inhibition of formation of brain lesions, and overall improvement in health. By using elevated doses of calcitriol in a pulsed regimen, particularly in a patient who has at least a normal level of serum 25-(OH)D₃, one avoids or substantially diminishes the incidence of hypercalcemia. In this way one can alleviate the symptoms of multiple sclerosis while avoiding the debilities associated with hypercalcemia. The pulsed elevated dose is found to be effective, where earlier efforts with human trials using repeated smaller doses resulted in hypercalcemia and required monitoring of the patients to ensure they avoided actions that could lead to hypercalcemia.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for inhibiting the development or progress of multiple sclerosis in a patient having multiple sclerosis or susceptible to the disabilities of multiple sclerosis, said method comprising: administering a dose of calcitriol-enhancing drug intermittently to said patient in an amount sufficient to inhibit the development or progress of multiple sclerosis and less than an amount to induce hypercalcemia.

2. A method according to claim 1, wherein said dose of calcitriol-enhancing drug is at least about 0.1 μg/kg calcitriol.

3. A method according to claim 2, wherein said calcitriol dose is in the range of about 0.1 to about 2 μg/kg.

4. A method according to claim 1, wherein said drug is calcitriol and said dose provides at least about 0.25 nmol/L calcitriol in the patient's blood.

5. A method according to claim 4, wherein said calcitriol dose provides a range from about 0.25 nmol/L to about 12 nmol/L calcitriol in the patient's blood.

6. A method according to claim 1, wherein the patient's blood level of 25-(OH)D₃ is maintained at least at about 50 nmol/L by exposure to sunlight or UVB light, by diet or by administration of supplements to enhance the amount of vitamin D₃ in the blood.

7. A method according to claim 6, wherein the patient's blood level of 25-(OH)D₃ is maintained in the range of about 85 nmol/L to about 120 nmol/L.

8. A method for inhibiting the occurrence of the symptoms of multiple sclerosis in a patient susceptible to brain lesions associated with multiple sclerosis, said method comprising: administering a calcitriol dose intermittently to said patient in an amount sufficient to inhibit the progress of multiple sclerosis and less than an amount to induce hypercalcemia, while maintaining a blood level of at least about 50 nmol/L 25-(OH)D₃ in the patient.

9. A method according to claim 8, wherein said intermittent administration is less frequently than weekly and more frequently than annually.

10. A method according to claim 8, wherein said drug is calcitriol and said dose provides at least about 0.25 nmol/L calcitriol in the patient's blood.

11. The method of claim 8, wherein said calcitriol dose provides a calcitriol level ranging from about 0.25 nmol/L to about 12 nmol/L in the patient's blood.

12. A method for inhibiting the progress of multiple sclerosis in a patient suffering from the disabilities of multiple sclerosis, said method comprising: administering intermittently a calcitriol dose in the range of about 0.1 to 2 μg/kg to said patient in an amount sufficient to inhibit the progress of multiple sclerosis and less than an amount to induce hypercalcemia.

13. A method according to claim 12, wherein said calcitriol dose provides at least about 0.25 nmol/L calcitriol in the patient's blood.

14. The method of claim 12, wherein said calcitriol dose provides a calcitriol level ranging from about 0.25 nmol/L to about 12 nmol/L in the patient's blood.

15. A method according to claim 12, wherein said intermittent administration is less frequently than weekly and more frequently than annually.

16. The method of claim 12, wherein vitamin D₃ is administered to maintain a 25-(OH)D₃ blood level in the range of about 85 to about 100 nmol/L.

17. The method of claim 12, wherein calcitriol is administered at an oral dose of about 0.5 μg/kg, and vitamin D₃ is administered after said calcitriol to maintain a 25-(OH)D₃ blood level at least about 85 nmol/L.

18. The method of claim 17 wherein the calcitriol is administered once every 5 to 10 days.

19. A composition comprising from about 0.1 to 2 μg/kg of calcitriol and an amount of vitamin D₃ sufficient to maintain a 25-(OH)D₃ blood level at least about 50 nmol/L.

20. The composition of claim 19, wherein the amount of calcitriol provides a range of about 0.25 nmol/L to about 12 nmol/L calcitriol in a patient's blood.
